(19) United States
(12) Patent Application Publication
(10) Pub. No.: US 2013/0143817 A1
(43) Pub. Date: Jun. 6, 2013

(54) USE OF TRANSFERRIN IN TREATMENT OF BETA-THALASSEMIAS

(71) Applicant: Yelena Z. Ginzburg, New York, NY (US)

(72) Inventor: Yelena Z. Ginzburg, New York, NY (US)

(73) Assignee: NEW YORK BLOOD CENTER, INC., New York, NY (US)

(21) Appl. No.: 13/809,848

(22) PCT Filed: Dec. 8, 2010
(86) PCT No.: PCT/US10/59543

§ 371 Date: Jan. 11, 2013

Related U.S. Application Data
(60) Provisional application No. 61/267,772, filed on Dec. 8, 2009.

(51) Int. Cl. A61K 38/40

(52) U.S. Cl. CPC A61K 38/40
USPC 514/13.5

(57) ABSTRACT

Disclosed herein are methods for treating disease, such as diseases of iron overload, including β-thalassemia, comprising administering at least one course of transferrin and thereby reducing the size of the spleen in said patient and reducing the concentration of iron in the tissues and blood.

### CROSS-REFERENCE TO RELATED APPLICATIONS

This application claims the benefit under 35 U.S.C. §119(e) to U.S. Provisional Patent Application No. 61/267,772 filed on Dec. 8, 2009, the entire contents of which are incorporated by reference herein.

### STATEMENT REGARDING FEDERALLY SPONSORED RESEARCH OR DEVELOPMENT

This invention was made with United States Government support of Grant Nos. HL68962 and HL07556 awarded by the National Institutes of Health/National Heart, Lung and Blood Institute. The United States Government may have certain rights in this invention.

### FIELD OF THE INVENTION

Disclosed herein are methods of treating β-thalassemias with transferrin.

### BACKGROUND

β-thalassemias are caused by mutations in the β-globin gene resulting in reduced or absent β-chain synthesis. A relative excess of α-globin chain synthesis leads to increased erythroid precursor apoptosis causing ineffective erythropoiesis, extramedullary expansion, and splenomegaly. Together with shortened red blood cell (RBC) survival, these abnormalities result in anemia. Patients with moderate or severe disease have increased intestinal iron absorption. Iron absorption, as well as iron recycling, is regulated by hepcidin; its binding to ferroportin (FPN-1) prevents iron egress from cells. Despite parenchymal iron overload in patients with β-thalassemia, hepcidin levels are low and do not appropriately increase in transfused patients with this disease. Relatively low levels of hepcidin mRNA expression in the liver are also characteristic of mouse models of β-thalassemia. This lack of an appropriate increase in hepcidin in β-thalassemia suggests that a competing signal is counter-regulating hepcidin expression despite increased parenchymal iron stores.

Mechanisms of hepcidin regulation are currently under investigation. While phlebotomy, anemia, hypoxia, and stimulation with erythropoietin lead to the suppression of hepcidin, in the absence of erythropoiesis, hepcidin suppression does not occur. Furthermore, hepcidin expression decreases in vitro when hepatocytes are exposed to sera from β-thalassemia patients as compared to control sera and increases when exposed to sera from recently transfused β-thalassemia patients as compared to sera from the same patients just prior to transfusion. In light of the central role hepcidin plays in iron metabolism, the lack of an appropriate increase in hepcidin expression suggests that a paradoxical state of iron deficient erythropoiesis, despite increased parenchymal iron stores, exists in (3-thalassemia. Hbb^{th1/th1} mice, the most commonly used murine model of β-thalassemia intermedia, when treated with iron have increased hemoglobin production resulting from an expansion of extramedullary erythropoiesis.

Transferrin functions as the main transporter of iron in the circulation where it exists in an iron-free apo-transferrin (apoTf) form, as monoferric transferrin, or as diferric holo-transferrin (holoTf). Typically, iron is bound to 30% of all transferrin binding sites in circulation. Transferrin-bound iron uptake by transferrin receptor 1 (TfR1) is the only known means of iron delivery for erythropoiesis. The effect of transferrin on erythropoietic iron delivery is greater than stoichiometric as the transfer of iron to cells results in repeated recycling of transferrin and the conversion of holoTf to apoTf for further iron binding and transport in circulation. In light of this, it is possible that the inability to compensate for the ineffective erythropoiesis and anemia observed in β-thalassemia is, in part, a consequence of an insufficient amount of circulating transferrin. Although transferrin expression is regulated by several factors, the degree of change is insufficient to accommodate the tremendous expansion of erythropoiesis and alteration in iron stores in β-thalassemia.

The current standard of care for treating diseases associated with inefficient erythropoiesis include red blood cell transfusions and iron chelation therapy. However, there are many downsides that accompany these current treatment methods, such as the risk of infection, development of red blood cell antibodies, iron overload, splenomegaly, and cost. Accordingly, there is a need for a method that simultaneously improves the efficiency of erythropoiesis and chelates iron from storage in the liver, spleen and heart of a subject without such unwanted side effects.

### SUMMARY

Disclosed herein are methods for decreasing iron deposition in an organ of a subject comprising administering to the subject an amount of transferrin effective to decrease iron deposition in the organ and methods of decreasing spleen size in patients with splenomegaly.

Disclosed herein is a method for reducing spleen size in a patient with thalassemia, the method comprising administering at least one course of transferrin doses to the patient and thereby reducing the size of the spleen in the patient. In one embodiment, the thalassemia is β-thalassemia intermedia or β-thalassemia major.

In one embodiment, the course comprises a plurality of doses of transferrin administered over a period of time from 7-21 days. In another embodiment, a dose of transferrin is administered every day during said course. In another embodiment, a dose transferrin is administered every other day during said course. In yet another embodiment, the course comprises administering transferrin every day for a certain number of days and every other day for a certain number of days. In yet another embodiment, the course is repeated at an interval selected form the group consisting of every other month, every third month, and every fourth month.

In one embodiment, each dose of transferrin comprises about 25-150 mg/kg of transferrin. In another embodiment, the transferrin is apotransferrin. In yet another embodiment, the transferrin is human transferrin.

### BRIEF DESCRIPTION OF THE DRAWINGS

FIG. 1 depicts normalized red blood cell (RBC) survival and appearance, less α-globin precipitation, and reduction of serum erythropoietin levels following transferrin injections in thalassemic mice. FIG. 1A: The half-life of RBC survival in apoTf- and holoTf-treated thalassemic mice was 37.1 and 37.0 days respectively, both representing significant improvement relative to baseline thalassemic mice (half-life of 10.1 days) (P<0.0001; n=5). FIG. 1B: Reduced α-globin deposition on RBC membranes in apoTf- and holoTf-treated thalassemic mice was observed in non-denaturing gel analysis. FIG. 1C: Morphology of RBCs in the peripheral smears was more normal with fewer reticulocytes, a higher density of RBCs, and less hypochromic cells in circulation (100× objective; n=10). FIG. 1D: Serum erythropoietin levels were reduced in transferrin-treated mice (n=5; *P<0.05).

FIG. 2 depicts that transferrin injections decrease splenomegaly, total spleen iron content, and extramedullary erythropoiesis in the liver while improving splenic architecture and shifting the proportion toward more mature erythroid precursors in the bone marrow and spleen of treated thalassemic mice. FIG. 2A: Reduction in the degree of splenomegaly was observed in transferrin-treated thalassemic mice, and correlated with more normal splenic architecture with more mature RBCs within the red pulp and a larger relative surface area devoted to white pulp (n=10). FIG. 2B: Spleen weight decreased after transferrin treatment (bars) and the amount of non-heme iron in the spleen was concurrently decreased (points) (n=5). FIG. 2C: In the bone marrow and spleen, transferrin injections overall lead to proportionally fewer immature and more mature erythroid precursors compared with untreated mice (n=5). (*P<0.0001; **P<0.05; ***P<0.001)

FIG. 3 depicts that transferrin injections result in a shift of apoptosis from late to early erythroid precursors. Flow cytometry determination of activated caspase 3 (A casp 3) was determined on cells gated for TER11/CD71. Mature erythroid precursor apoptosis decreased while immature erythroid precursor apoptosis increased, particularly in the spleen of transferrin-treated thalassemic mice (n=5) (*P<0.0001; **P<0.05 relative to untreated thalassemic mice).

FIG. 4 depicts increases in liver hepcidin expression after transferrin injections with a concurrent decrease in ferroportin (FPN-1) in transferrin-treated thalassemic mice. FIG. 4A: Hepcidin expression increased in the livers of transferrin-treated thalassemic mice. FIG. 4B: Liver sections were stained with FPN-1 antibody and counterstained with Perls' Prussian blue. FPN-1 was positive on fewer Kupffer cells in the livers of transferrin-treated mice, whereas iron was observed by Perls' Prussian blue staining in a similar number of cells (20× objective; n=5). (*P<0.0001)

FIG. 5 depicts spleen size and erythropoietin levels in transferrin-treated thalassemic mice. No change in spleen size (FIG. 5A) or erythropoietin levels (FIG. 5B) were observed after 10 days of daily IP transferrin injections in thalassemic mice. Although the weight of the spleen in untreated thalassemic mice is greater than that of WT mice, 10 days of transferrin injections did not result in a change in spleen weight in treated thalassemic mice. FIG. 5B: Although serum erythropoietin levels were significantly higher in untreated thalassemic as compared to WT mice, no change in these levels was measurable after 10 days of transferrin injections in treated thalassemic mice. Erythroid proliferation and erythroid drive are affected in a feedback loop secondary to other more direct effects of exogenous transferrin. (*P<0.0001; **P<0.05; IP=intraperitoneal; WT=wild type C57BL/6J; apoTf=apotransferrin; holoTf=holotransferrin).

FIG. 6 depicts WT mice treated with transferrin for 60 days. These mice did not exhibit many of the changes observed in transferrin-treated thalassemic mice. There was no change in spleen weight (FIG. 6A), RBC survival (FIG. 6B), degree of α-globin precipitation of RBC membranes (FIG. 6C), serum erythropoietin levels (FIG. 6D), or hepcidin expression in the liver (FIG. 6E) observed in transferrin-treated WT mice as compared to untreated mice. This data suggests that the effect of exogenous transferrin in thalassemic mice is disease specific (*P<0.0001; n=5).

FIG. 7 depicts that transferrin-treated WT mice exhibit changes in number of erythroid precursors and erythroid precursor apoptosis similar to that observed in transferrin-treated thalassemic mice. FIG. 7A: No decrease in immature erythroid precursors was seen in the bone marrow or spleen of transferrin-treated WT mice as compared to untreated mice. A higher proportion of mature erythroid precursors were found in the spleen. FIG. 7B: Like transferrin-treated thalassemic mice, apoptosis of mature erythroid precursors decreased in the bone marrow and spleen of transferrin-treated WT mice as compared to untreated mice. Exogenous transferrin has either a direct effect on rates of erythroid precursor apoptosis or indirectly results in these changes by changing the amount of hemoglobin per cell (*P<0.0001; **P<0.05; n=5).

FIG. 8 depicts iron saturation (FIG. 8A) and LPI (FIG. 8B) in apotransferrin-treated and untreated splenectomized thalassemic mice.

FIG. 9 depicts extramedullary erythropoiesis in the liver of apotransferrin-treated and untreated splenectomized thalassemic mice.

FIG. 10 depicts serum erythropoietin concentrations in apotransferrin-treated and untreated splenectomized thalassemic mice.

FIG. 11 depicts RBC morphology in apotransferrin-treated and untreated splenectomized thalassemic mice.

FIG. 12 depicts RBC lifespan in untreated (FIG. 12A) and apotransferrin-treated (FIG. 12B) splenectomized thalassemic mice.

FIG. 13 depicts α-globulin precipitation in untreated and apotransferrin-treated splenectomized thalassemic mice.

FIG. 14 depicts cytospin (FIG. 14A) and flow cytometric (FIG. 14B) analysis of erythroid differentiation in apotransferrin-treated thalassemic mice pre- and post-splenectomy.

### DETAILED DESCRIPTION

Disclosed herein are methods for treating diseases of ineffective erythropoiesis by administration of transferrin. Transferrin-bound iron is the major source of iron for erythropoiesis, therefore increasing the quantity of circulating transferrin compensates for ineffective erythropoiesis. Exogenous transferrin improves the efficiency of erythropoiesis and decreases volatile iron species leading to an increased number of circulating red blood cells (RBCs) and hemoglobin (Hb), normalized red blood cell survival in circulation, reduced reticulosytosis, reversed splenomegaly, and decreased concentration of labile plasma iron (LPI).

The data presented herein reveals that there is a significantly higher LPI level in untreated thalassemic mice relative to WT mice despite no difference in transferrin saturation. Transferrin injections result in normalization of LPI levels in thalassemic mice. Increased iron turn over in β-thalassemia results in local disequilibrium in which the amount of iron recycled and released exceeds the amount of locally available apoTf. Other serum proteins, the most abundant of which is albumin, are able to bind iron in such conditions, albeit with lower affinity. Albumin-bound iron is redox active and thus still considered LPI but is unavailable for binding to apoTf.

RBC parameters improved in transferrin-treated relative to untreated thalassemic mice directly or indirectly as a result of reduced α-globin precipitation in thalassemic RBC membranes. Because α-globin membrane precipitation has been associated with shortened RBCs survival, transferrin-treatment reversed this process resulting in a normalized RBC survival and a consequent increase in the total number of circulating RBCs. Exogenous transferrin may enable iron delivery to a greater number of erythroid precursors while decreasing the net iron availability per cell. In support of this is the demonstration that monoferric transferrin is the predominant form of transferrin in circulation when transferrin saturation is lowered and that each molecule of monoferric transferrin delivers less iron than holoTf. Furthermore, K562 cells in culture exposed to excess apo-transferrin exhibit a reduction in cytoplasmic iron in a dose response manner. Because of these factors and the fact that iron delivery for erythropoiesis is limited to transferrin-bound iron, exogenous transferrin enables the delivery of iron to more erythroid precursors, resulting in a greater number of mature RBCs, each with less heme and Hb, resulting in a lower mean corpuscular hemoglobin (MCH), but because of a higher mean corpuscular hemoglobin concentration (MCHC), an increase in total circulating Hb.

The increase in Hb and hematocrit (HCT) in transferrin-treated thalassemic mice results in a feedback reduction in reticulocyte count, erythropoietin levels, and splenomegaly. Because splenomegaly itself is often implicated in worsening anemia, its reversal is a possible cause of improved Hb. However, the 10 day injection experiments presented herein resulted in increased Hb despite having no effect on spleen size. In this disease the (enlarged) spleen has the dual role of being the site for extramedullary erythropoiesis as well as erythroid precursor apoptosis. Splenomegaly in transferrin-treated mice was reversed as a consequence of a combination of factors: 1) decreased extramedullary erythropoiesis resulting from decreased serum erythropoietin levels, and 2) decreased mature erythroid precursor apoptosis, secondary to decreased α-globin precipitation. The large spleen in β-thalassemia may serve as a reservoir for quiescent immature erythroid precursors that do not mature, yet as a consequence of elevated erythropoietin levels, cannot undergo apoptosis.

Mice treated for 60 days and those treated for 10 days with transferrin displayed a different distribution of non-heme iron. This implies that, although there is an initial shift of iron stores out of the liver and heart, ultimately, non-heme iron from the spleen is used to expand the number of RBCs in circulation in transferrin-treated mice. Exogenous transferrin diminished non-heme iron distribution in this model by shifting iron from parenchyma to the circulating Hb compartment.

Mice exhibit no evidence of toxicity to human transferrin injection, and survive the course of injections without ill-effects. In prior studies using human transferrin in mice, no increase in the number of circulating CD4⁺ or CD8⁺ T cells was demonstrated. Mouse anti-human transferrin antibody ELISA was performed and these antibodies were detected in sera of transferrin-treated mice. Despite this immune response, a robust improvement in disease physiology was observed. The use of same-species transferrin injection could potentially lead to even greater improvements in Hb.

An increase in hepcidin expression and a concurrent decrease in FPN-1 levels after transferrin treatment would benefit β-thalassemia patients by preventing further iron release into circulation. Increased hepcidin expression despite low transferrin saturation in transferrin-treated mice with decreased extramedullary erythropoiesis provides further evidence for the existence of an “erythroid regulator” of hepcidin. One possible regulator is growth differentiation factor 15 (GDF15) which is dramatically elevated in patients with β-thalassemia. A recent follow-up study demonstrated that another factor, twisted gastrulation, is expressed at significantly higher levels in β-thalassemic mice and resulted in a dose-responsive suppression of hepcidin in primary mouse hepatocyte cultures. It has been proposed that GDF15 and twisted gastrulation act together to inhibit hepcidin in β-thalassemia. Further evaluation of these factors is needed to identify changes in expression that may explain the mechanisms underlying increased hepcidin levels in transferrin-treated mice.

Human transferrin may have several other potential uses including, but not limited to, treatment of patients with diseases of concurrent anemia and iron overload. Examples of such diseases include β-thalassemia and myelodysplastic syndromes. In these circumstances, additional transferrin could be used to abrogate ineffective erythropoiesis by redirecting iron from storage and parenchymal deposition to erythropoietic machinery for Hb synthesis. The safety of human transferrin injections has already been demonstrated. β-thalassemia intermedia is the human disease closest to the thalassemic mice used in these experiments, making patients with β-thalassemia intermedia, as well as β-thalassemia major, the natural population for use of the disclosed compositions and methods. These methods are suitable for splenectomized and non-splenectomized patients. A novel approach would greatly benefit this patient population for whom standard management has consisted of transfusion followed by chelation therapy for the last half-century.

Management of patients with β-thalassemia intermedia (TI) is less standardized compared to treatment of β-thalassemia major (TM). The reason for this has to do with the limited ability of physicians to accurately define TI as well as the dearth of available treatment options. TI patients are typically characterized as those who are transfusion-independent with a clinical course intermediate in severity between TM and asymptomatic heterozygotes (carriers). TI patients have homozygous mutations as do those with TM but a relatively milder course or greater ability to synthesize hemoglobin due to disease modifiers. The spectrum of disease in patients with TI is wide, ranging from those able to produce 6 g/dL of hemoglobin (and require only occasional or intermittent transfusions) at the expense of huge hematopoietic expansion and skeletal abnormalities to those who are completely asymptomatic with mild anemia and splenomegaly. The management of TI would be more similar to that of TM if alternatives to chronic transfusion were available.

The survival, quality of life, and sexual maturation is higher in TI relative to TM patients suggesting that the complications of chronic transfusions may outweigh the benefit, certainly in patients who continue to grow and thrive between the second and third year without them. In a series of 165 TI patients, most (95%) were diagnosed after age two years, a majority (60%) started requiring at least occasional transfusions between two and five years of age, and many (28%) become transfusion-dependent in adulthood (Kazazian HH 1990 Seminars Hematology). Children who require only intermittent transfusions but are only able to maintain reasonable levels of hemoglobin as a consequence of extensive hematopoietic expansion often develop hypersplenism that may exacerbate their anemia, and benefit from splenectomy.

Surgical splenectomy is typically the first therapeutic approach considered to correct anemia before starting regular transfusions although the age at splenectomy in TI is older than in TM. This surgical procedure enables the patient to also have a liver biopsy to assess iron status, although polyvalent pneumococcal vaccine is mandatory to avoid overwhelming infection. Children require penicillin prophylaxis against pneumococcal infections following splenectomy, and although not supported by clinical trials, asplenic adults with non-specific febrile illness are regularly treated early with antibiotics. Taken together, splenectomy is temporarily effective in reversing anemia or delaying/lowering transfusion need. Although it is relatively preferred to other options, the potentially life-threatening consequences make this procedure less than optimal.

Administration of exogenous transferrin in diseases associated with ineffective erythropoiesis results in a “non-surgical” splenectomy with reversal of splenomegaly in transferrin-treated Hbb^{th1/th1} mice. As used herein “non-surgical splenectomy” refers to reduction in spleen size accomplished by non-surgical means, such as by administration of transferrin. The typical consequence of surgical splenectomy in mice is anemia (especially as the mice age) and/or extramedullary erythropoiesis in the liver. Non-surgical splenectomy results from higher hemoglobin concentration, more red blood cells, and a normal red blood cell survival, making it a consequence of more efficient erythropoiesis, confirmed by fewer reticulocytes, reduced serum erythropoietin, and an increased proportion of mature relative to immature erythroid precursors in the bone marrow and spleen of transferrin-treated Hbb^{th1/th1} mice. Non-surgical splenectomy is not associated with increased risk of infection as surgical splenectomy. Surprisingly, transferrin injections in surgically splenectomized mice resulted in improved survival, likely due to reversal of anemia.

In one embodiment of the above-described methods, the subject can be a mammal, such as a mouse, rat, cat, dog, horse, sheep, cow, steer, bull, livestock, or monkey or other primate. In the preferred embodiment, the subject is a human.

In one embodiment of the above-described methods, the transferrin is human transferrin, either transferrin isolated from human blood or recombinant human transferrin. In additional embodiments, the transferrin is apotransferrin or holotransferrin.

In accordance with the methods disclosed herein, the transferrin may be administered to a human or other animal subject by known procedures, including, without limitation, nasal administration, oral administration, parenteral administration (e.g., epidural, epifascial, intracapsular, intracutaneous, intradermal, intramuscular, intraorbital, intraperitoneal, intrasternal, intravascular, intravenous, parenchymatous, and subcutaneous administration), sublingual administration, transdermal administration, and administration by osmotic pump. Preferably, transferrin is administered via intraperitoneal, intravenous or intramuscular injection.

In one embodiment, transferrin is administered by intravenous infusion over a period of time, such as from 15 minutes to 2 hours or 30 minutes to 1 hour. Methods for intravenous infusion of transferrin are known to persons of ordinary skill in the art, such as physicians, and can be implemented by such persons according to the patient's individual needs.

In accordance with the methods disclosed, proper dosages of transferrin can be determined without undue experimentation using standard dose-response protocols. Exemplary doses of transferrin for human administration in accordance with the disclosure herein are from 25-150 mg/kg, 50-125 mg/kg, 75-100 mg/kg, or 85-115 mg/kg. These doses of transferrin are well tolerated without serious adverse events in this relatively ill patient population.

The transferrin can be administered, for example, daily, weekly, monthly or annually. Exemplary dosing regimens (courses) include, but are not limited to, daily for 7-21 days, daily for 10-14 days, every other day for 7-21 days, every other day for 10-14 days, every other day for 14-21 days, every other day for 14 days, every day for 10 days. Courses can also comprise dosing regimens wherein certain doses are administered at one interval and additional doses are administered at a second interval. For example, and not intended to be a limiting example, transferrin is administered daily for three days and then every other day for 10 days. Additionally, a course can be repeated periodically, for example, monthly, every other month, every three months, every four months, every five months or every six months. Courses can be repeated indefinitely.

In additional embodiments, each course can use the same or different doses of transferrin.

### EXAMPLES

### Example 1

### Exogenous Human Transferrin is Functional in Mouse Circulation

Human, rather than mouse, transferrin was selected for injection because it enabled analysis of the quantities of each type of transferrin separately. Daily injections were employed in light of the 34-40 hr half-life of endogenous transferrin in mice and on the basis of prior experiments in hypotransferrinemic mice. The injected transferrin was in either the apotransferrin or holotransferrin form. The optimum dose of 10 mg transferrin per day was determined by dose escalation experiments (data not shown). Because maturation of committed precursors from erythroid colony-forming unit (CFU-E) stage to normoblast stage typically takes 7-10 days, initially mice treated with transferrin for 10 days were analyzed. However, as no effects on spleen size or serum erythropoietin levels were seen by 10 days, a 60 days course was selected to represent a more chronic state of increased transferrin in the circulation.

The degree of transferrin saturation, the total iron binding capacity (TIBC), and the mouse transferrin and LPI concentrations were examined before and after human transferrin injection into thalassemic and WT mice. Although no differences in TIBC or transferrin saturation were found between untreated thalassemic and WT mice, LPI levels were higher in thalassemic mice (Table 1). Transferrin injection increased the TIBC and decreased the transferrin saturation in both WT and thalassemic mice and returned LPI levels to normal in the circulation of thalassemic mice (Tables 1 and 2). Transferrin injection also resulted in the suppression of endogenous transferrin production in thalassemic mice. The sum of the endogenous and exogenous transferrin concentrations yielded an approximately twofold increase in circulating transferrin concentration in transferrin-treated WT and thalassemic mice relative to untreated mice. The effects of apotransferrin and holotransferrin injection were similar in all analyses. These results show that high levels of transferrin can be reached in circulation, resulting in lowered LPI concentration in transferrin-treated thalassemic mice; this result indicated that the injected transferrin retains iron-binding capacity.

TABLE 1
Transferrin concentrations and iron parameters in transferrin-treated thalassemic mice

| | | | | Human | Mouse |
|---|---|---|---|---|---|
| | | Transferrin | | transferrin | transferrin |
| | TIBC | saturation | | concentration | concentration |
| | (μg dl⁻¹) | (%) | LPI (μM) | (mg ml⁻¹) | (mg ml⁻¹) |
| Untreated | 410.8 ± 19.4 | 33.7 ± 2.1 | 0.03 ± 0.01 | 0 | 2.9 ± 0.3 |
| WT mice | | | | | |
| Untreated | 415.2 ± 11.3 | 31.5 ± 2.8 | 0.27 ± 0.06 | 0 | 2.8 ± 0.2 |
| thalassemic | (P = 0.85) | (P · 0.67) | (P = 0.0001) | | (P = 0.95) |
| mice | | | | | |
| ApoTf- | 595.8 ± 29.4 | 21.5 ± 1.8 | 0.02 ± 0.01 | 2.7 ± 0.2 | 2.3 ± 0.2 |
| treated | (P < 0.0001) | (P = 0.02) | (P = 0.001) | (P < 0.0001) | (P = 0.02) |
| thalassemic | | | | | |
| mice | | | | | |
| HoloTf- | 597.0 ± 20.7 | 20.1 ± 1.4 | 0.02 ± 0.05 | 2.7 ± 0.2 | 2.1 ± 0.1 |
| treated | (P < 0.0001) | (P. = 0.005) | (P = 0.01) | (P < 0.0001) | (P = 0.0006) |
| thalassemic | | | | | |
| mice | | | | | |

Data represent means ± s.e.m.

TABLE 2
Transferrin concentrations and iron parameters in transferrin-treated WT mice

| | | | | Human | Mouse |
|---|---|---|---|---|---|
| | | Transferrin | | transferrin | transferrin |
| | TIBC | saturation | | concentration | concentration |
| | (μg dl⁻¹) | (%) | LPI (μM) | (mg ml⁻¹) | (mg ml⁻¹) |
| Untreated | 410.8 ± 19.4 | 33.7 ± 2.1 | 0.03 ± 0.01 | 0 | 2.2 ± 0.1 |
| WT mice | | | | | |
| ApoTf- | 658 ± 19 | 14.2 ± 0.7 | −0.02 ± 0.03 | 2.8 ± 0.1 | 2.3 ± 0.1 |
| treated WT | | (P = 0.006) | (P = 0.21) | (P < 0.0001) | (P = 0.28) |
| mice | | | | | |
| HoloTf- | 601.5 ± 12 | 16.0 ± 0.8 | −0.21 ± 0.01 | 2.5 ± 0.2 | 2.2 ± 0.0 |
| treated WT | (P = 0.0001) | (P = 0.002) | (P < 0.0001) | (P < 0.0001) | (P = 0.81) |
| mice | | | | | |

(n = 5 per group; TIBC = total iron binding capacity; IP = intraperitoneal; LPI = labile plasma iron; WT = wild type C57BL/6J).

### Example 2

### Anemia is Partially Reversed with More Red Cells and Fewer Reticulocytes

Compared to untreated thalassemic mice, transferrin-treated thalassemic mice showed a higher number of RBCs, more abundant hemoglobin and an increased hematocrit, as well as lower reticulocyte counts (Table 3). The higher total hemoglobin abundance in transferrin-treated thalassemic mice can be accounted for by the higher number of RBCs plus the increased MCHC; MCHC refers to the average concentration of hemoglobin within RBCs, calculated by dividing mean corpuscular volume (MCV) by mean corpuscular hemoglobin (MCH). MCV and MCH refer respectively to the average size of RBCs and the amount of hemoglobin contained per RBC. Although both MCV and MCH decreased as a consequence of transferrin treatment, MCV decreased to a greater extent than MCH, resulting in a higher MCHC (Table 3).

Furthermore, the decreased reticulocyte counts in transferrin-treated relative to untreated thalassemic mice correlate well with the finding of a decreased RBC distribution width (Table 3), indicating that the variability in cell size is closer to normal, and with the finding of a reduction in MCV, because reticulocytes are typically larger than mature RBCs. Again, the effects of apotransferrin and holotransferrin injection were similar and were observed as early as 10 days after treatment (data not shown). Although transferrin injection of WT mice did not result in an increase in hemoglobin concentrations and hematocrit, this treatment did increase the number of circulating RBCs and lowered MCV, MCH, MCHC, CH^{r} and CH^{m} (Table 4); CH^{r} and CH^{m} are measures of MCH in reticulocytes and mature RBCs, respectively. Because transferrin-treated WT mice showed a decrease in MCH despite an increase in reticulocyte count, the lower MCH in transferrin-treated thalassemic mice is secondary to an intrinsic effect of the injected transferrin on cell hemoglobin synthesis.

TABLE 3
RBC parameters in transferrin-treated thalassemic mice reveal a significant increase in
RBC number, Hb, and HCT with a decrease in reticulocyte count, MCV, and MCH
compared to untreated mice.

| | RBC | Hb | HCT | MCV | MCH |
|---|---|---|---|---|---|
| | (×10⁶ cells/L) | (g/dL) | (%) | (fL) | (pg) |
| Untreated | 10.0 ± 0.2 | 13.1 ± 0.4 | 42.3 ± 0.9 | 42.3 ± 0.3 | 13.4 ± 0.2 |
| WT | | | | | |
| Untreated | 8.9 ± 0.2* | 8.2 ± 0.2* | 30.2 ± 0.4* | 34.0 ± 0.8* | 9.3 ± 0.3* |
| thalassemic | | | | | |
| mice | | | | | |
| ApoTf-treated | 15.1 ± 0.3** | 11.4 ± 0.2** | 36.9 ± 0.7** | 24.6 ± 0.3** | 7.5 ± 0.1** |
| thalassemic | | | | | |
| mice | | | | | |
| HoloTf- | 14.7 ± 0.3 | 11.8 ± 0.1 | 38.2 ± 0.4** | 26.0 ± 0.4** | 8.0 ± 0.1 |
| treated | | | | | |
| thalassemic | | | | | |
| mice | | | | | |

| | MCHC | Retics | CH^{r} | CH^{m} | RDW |
|---|---|---|---|---|---|
| | (g/dL) | (×10⁹ cells/L) | (pg) | (pg) | (%) |
| Untreated | 31.6 ± 0.5 | 299 ± 20 | 13.3 ± 0.2 | 12.4 ± 0.1 | 16.1 ± 0.1 |
| WT | | | | | |
| Untreated | 27.5 ± 0.4* | 2325 ± 168* | 11.5 ± 0.2* | 9.6 ± 0.1* | 36.7 ± 0.4* |
| thalassemic | | | | | |
| mice | | | | | |
| ApoTf-treated | 30.7 ± 0.1** | 846 ± 26** | 9.2 ± 0.1** | 7.9 ± 0.1** | 25.6 ± 0.2** |
| thalassemic | | | | | |
| mice | | | | | |
| HoloTf- | 30.9 ± 0.2 | 1107 ± 67** | 9.4 ± 0.1** | 8.2 ± 0.1** | 26.6 ± 0.5** |
| treated | | | | | |
| thalassemic | | | | | |
| mice | | | | | |

Data represents mean s.e.m.
*P < 0.0001 versus untreated WT mice;
**P < 0.0001 versus untreated thalassemic mice.
CH^{r}, reticulocyte hemoglobin content;
CH^{m}, mature RBC hemoglobin content;
RDW, RBC distribution width.

TABLE 4
Transferrin-treated WT mice have more RBCs, lower MCV, MCH, and MCHC with an increased reticulocyte count after 60 days of
daily IP injections.

| | RBC | | | | | | Retics | | | |
|---|---|---|---|---|---|---|---|---|---|---|
| | (×10⁶ | Hb | HCT | MCV | MCH | MCHC | (×10⁹ | CH^{r} | CH^{m} | RDW |
| | cells/L) | (g/dL) | (%) | (fL) | (pg) | (g/dL) | cells/L) | (pg) | (pg) | (%) |
| Untreated | 10.5 ± 0.2 | 15.1 ± 0.4 | 44.3 ± 1.0 | 42.1 ± 0.2 | 14.3 ± 0.1 | 34.0 ± 0.3 | 225 ± 11 | 14.6 ± 0.2 | 14.1 ± 0.1 | 12.5 ± 0.1 |
| WT mice | | | | | | | | | | |
| ApoTf- | 14.2 ± 0.1 | 13.6 ± 0.1 | 43.9 ± 0.1 | 30.9 ± 0.4 | 9.6 ± 0.1 | 31.1 ± 0.1 | 463 ± 34 | 10.9 ± 0.1 | 9.6 ± 0.1 | 16.6 ± 0.2 |
| treated | (P < 0.0001) | (P = 0.03) | (P = 0.81) | (P < 0.0001) | (P < 0.001) | (P < 0.0001) | (P < 0.0001) | (P < 0.0001) | (P < 0.0001) | (P < 0.0001) |
| WT mice | | | | | | | | | | |
| HoloTf- | 14.0 ± 0.1 | 14.3 ± 0.2 | 45.8 ± 0.5 | 32.7 ± 0.4 | 10.2 ± 0.0 | 31.1 ± 0.1 | 355 ± 23 | 11.0 ± 0.2 | 10.2 ± 0.1 | 15.3 ± 0.2 |
| treated | (P < 0.0001) | (P = 0.12) | (P = 0.23) | (P < 0.0001) | (P < 0.0001) | (P < 0.0001) | (P < 0.0001) | (P < 0.0001) | (P < 0.0001) | (P < 0.0001) |
| WT mice | | | | | | | | | | |

n = 5 per group;
wt = wild type C57BU6J;
RBC = red blood cell;
MCV = mean corpuscular volume;
MCH = mean corpuscular hemoglobin;
MCHC = mean corpuscular hemoglobin concentration;
IP = intraperitoneal

These findings in transferrin-treated thalassemic and WT mice suggest that transferrin injection results in a state of iron-restricted-like erythropoiesis. Typically, as in the case of iron-deficiency anemia, iron-restricted erythropoiesis is associated with low MCV and MCH values in which the amount of heme and hemoglobin per cell is low as a consequence of less iron delivery to each erythroid precursor, and fewer cells are made, resulting in a low-MCV anemia. Thalassemic mice were able to benefit from the reduction in MCV and MCH caused by transferrin treatment, which resulted in less α-globin precipitation on RBC membranes and consequently increased RBC survival and a greater number of circulating RBCs. Because β-thalassemia is associated with a disparity of α- and β-globin production and because globin production is transcriptionally regulated by heme, a decrease in heme synthesis would be expected to result in less α-globin precipitation on RBC membranes. The results obtained with transferrin injection into WT mice show that additional transferrin has the inherent ability to apportion smaller doses of iron into a greater number of RBCs.

### Example 3

### Normalized RBC Survival Leads to a Reduction of Serum Erythropoietin

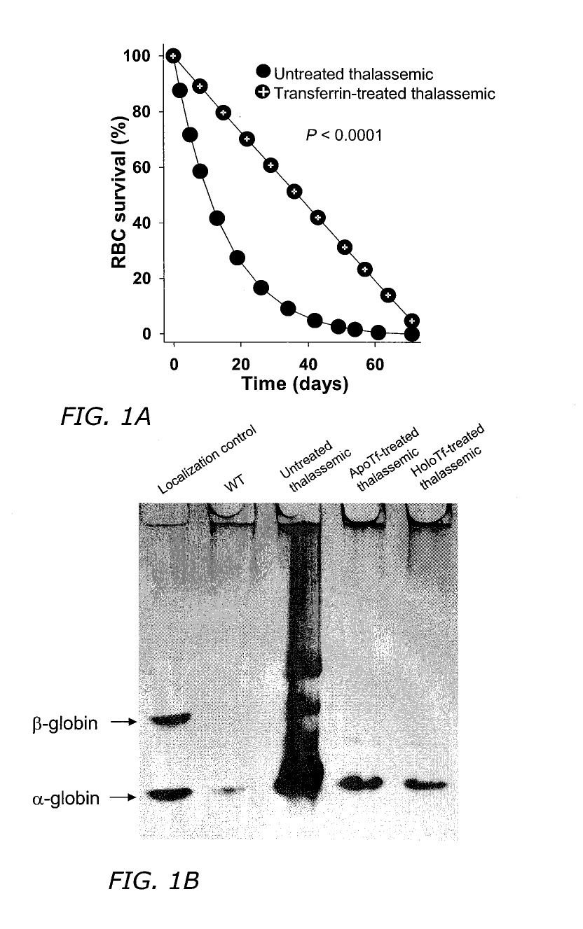

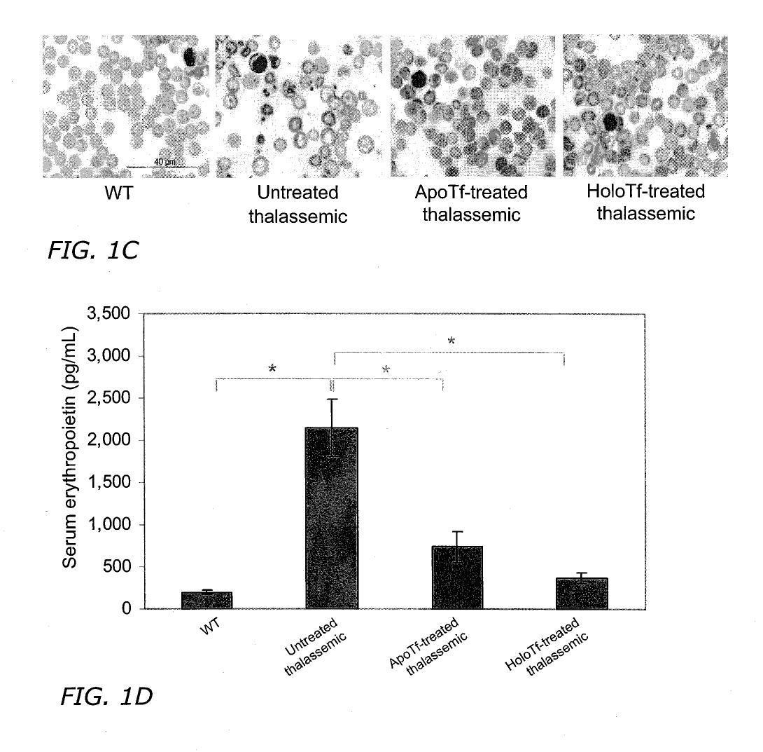

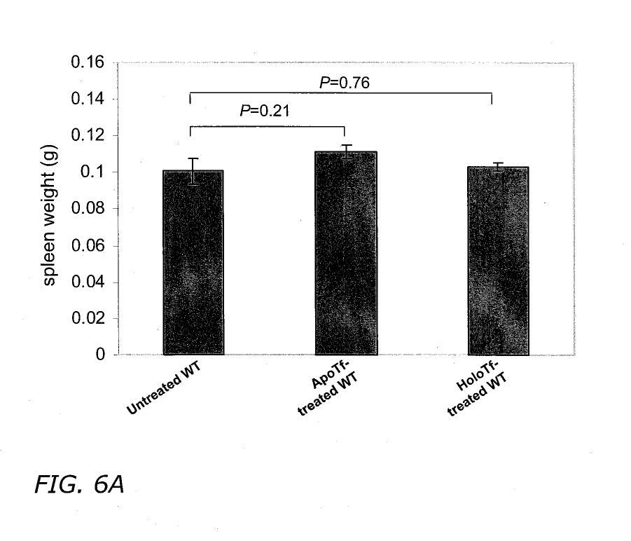

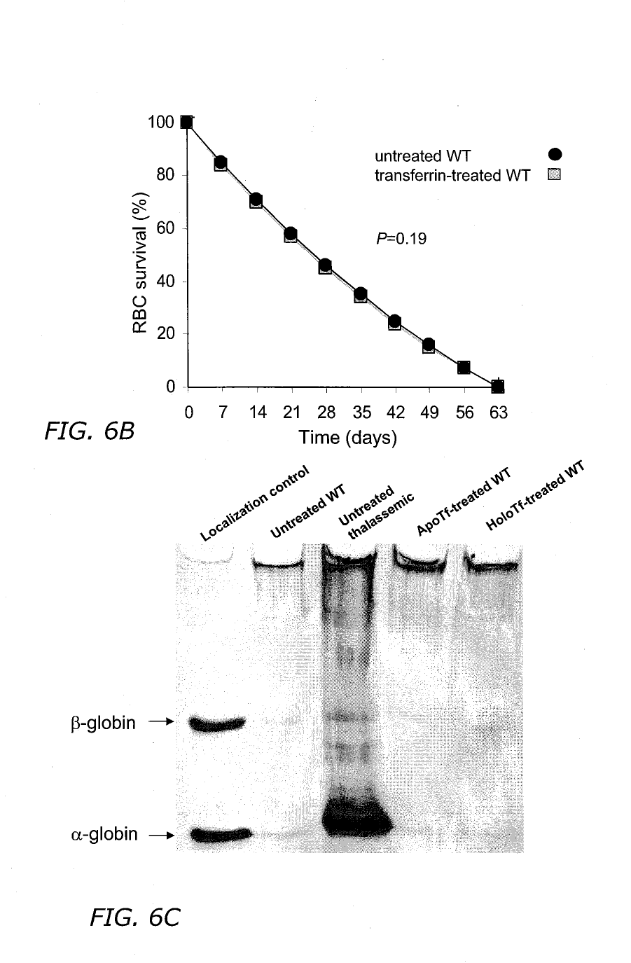

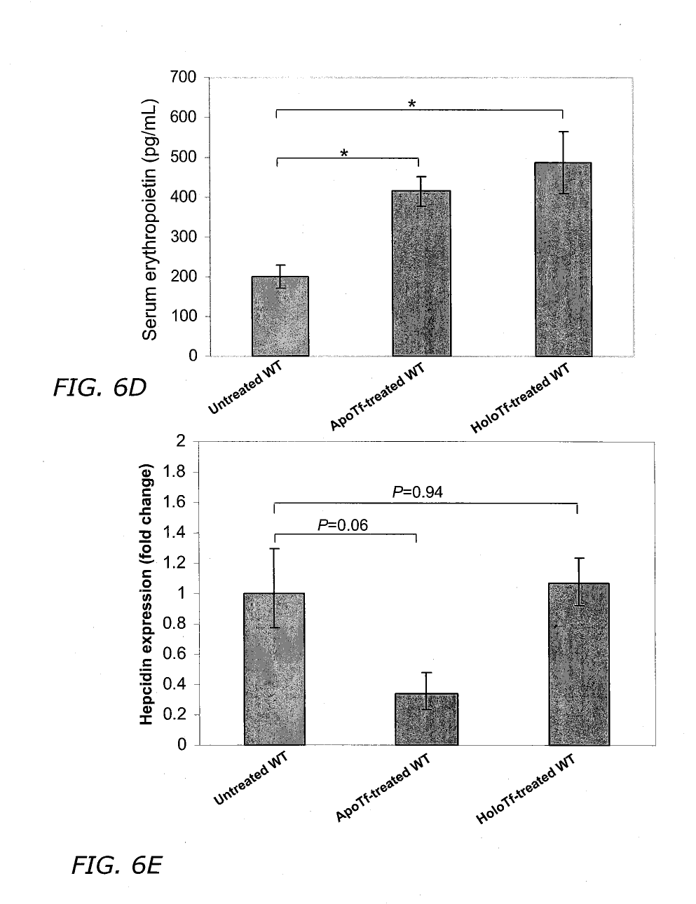

A shortened RBC survival time was observed in a mouse model of 13-thalassemia intermedia similar to the model used herein, and a similar effect was observed by the present inventors in thalassemic mice compared with WT mice (data not shown). Treatment with transferrin normalized RBC survival in thalassemic mice; the half-life of RBC survival in apotransferrin (ApoTf) and holotransferrin (HoloTf)-treated thalassemic mice was 37.1 and 37.0 days, respectively, representing significant improvement relative to baseline thalassemic mice (half-life of 10.1 days) (FIG. 1A). This finding correlates with a decrease in the amount of α-globin precipitates on RBC membranes of apotransferrin- and holotransferrin-injected thalassemic mice (FIG. 1B) and for the increased number of RBCs in the circulation of transferrin-treated mice. Neither of these findings was observed in transferrin-treated WT mice (FIGS. 6B and C). In further support of the beneficial effects of transferrin, the severity of erythrocyte morphological abnormalities, as assessed in peripheral blood smears, was markedly ameliorated (FIG. 1C).

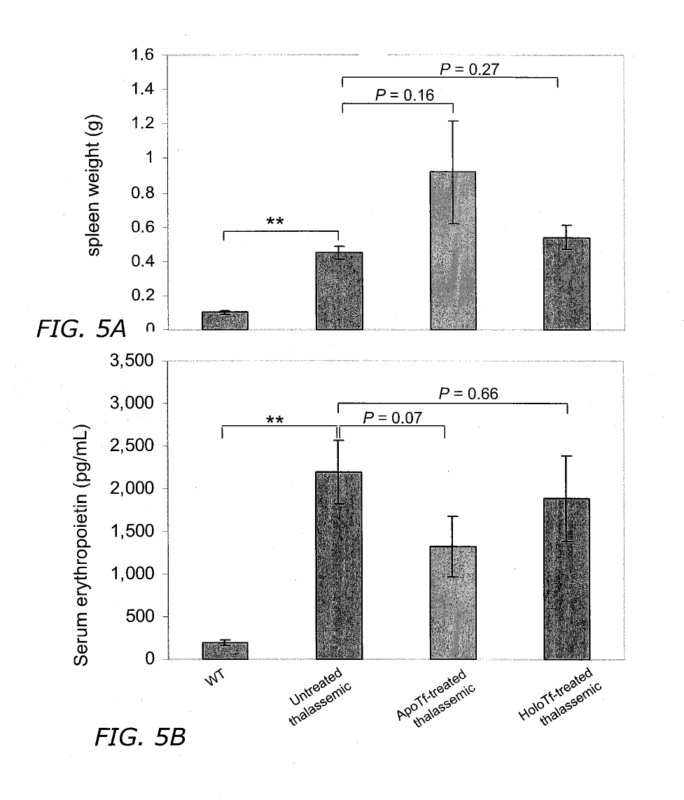

Serum erythropoietin concentrations in apotransferrin- and holotransferrin-treated thalassemic mice were decreased, likely resulting from feedback regulation due to the increased number of circulating RBCs and increased hemoglobin concentration (FIG. 1D). No decrease in serum erythropoietin concentration was observed in transferrin-treated WT mice (FIG. 6D). Although a 10 day course of transferrin injections into thalassemic mice was able to normalize RBC survival, reduce α-globin membrane precipitation and increase circulating RBC counts and hemoglobin concentration (data not shown), no change in erythropoietin concentration was observed at this time point (FIG. 5B), suggesting that this feedback process is not direct.

### Example 4

### Reversed Splenomegaly and Improved Erythroid Precursor Maturation

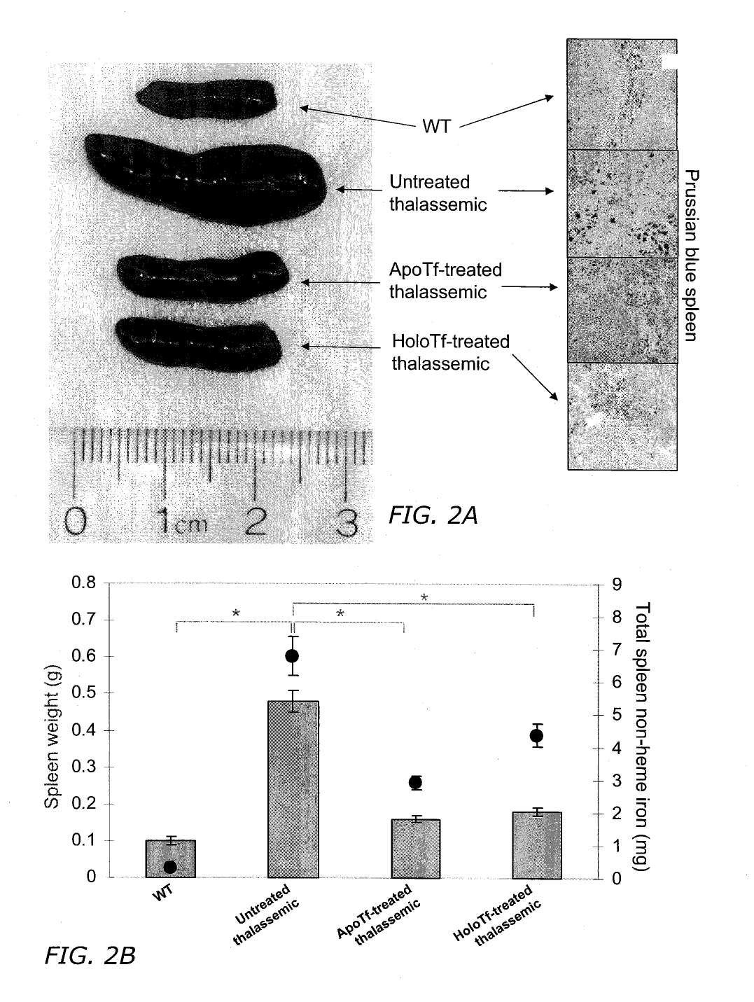

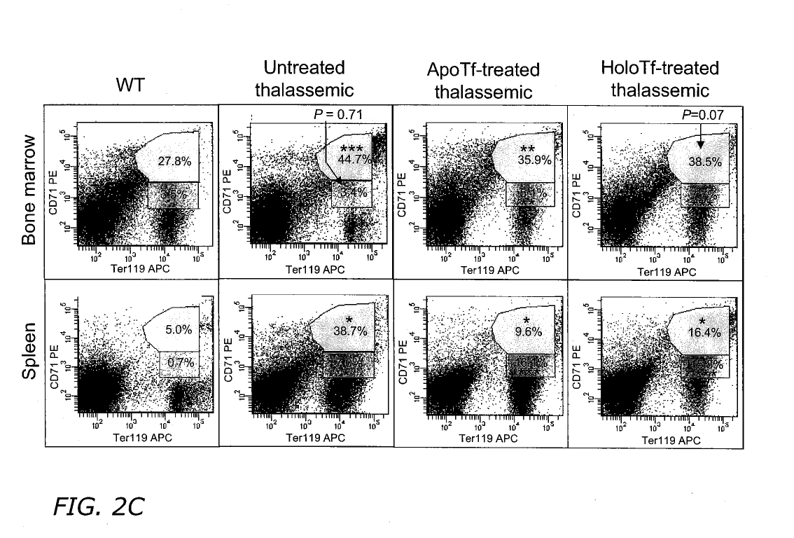

Transferrin injection resulted in a marked reduction in spleen size (FIG. 2A) and weight (FIG. 2B) with more organized splenic architecture containing larger germinal centers and less red pulp compared with untreated thalassemic mice (FIG. 2A). This effect was not observed in transferrin-treated WT mice (FIG. 6A) or after the 10 day course of transferrin injection in thalassemic mice (FIG. 5A). There are more erythroid precursors in untreated thalassemic mice relative to WT mice in both bone marrow and spleen, as assessed by flow cytometry. Thus, these current findings suggest that transferrin injection into thalassemic mice reduces extramedullary erythropoiesis in the spleen. Moreover, there was a considerable reduction in extramedullary erythropoiesis in the liver as determined by immunohistochemistry staining of liver sections with antibodies to TER119 (data not shown).

### Example 5

### Pattern of Apoptosis Favors More Mature Erythroid Precursors

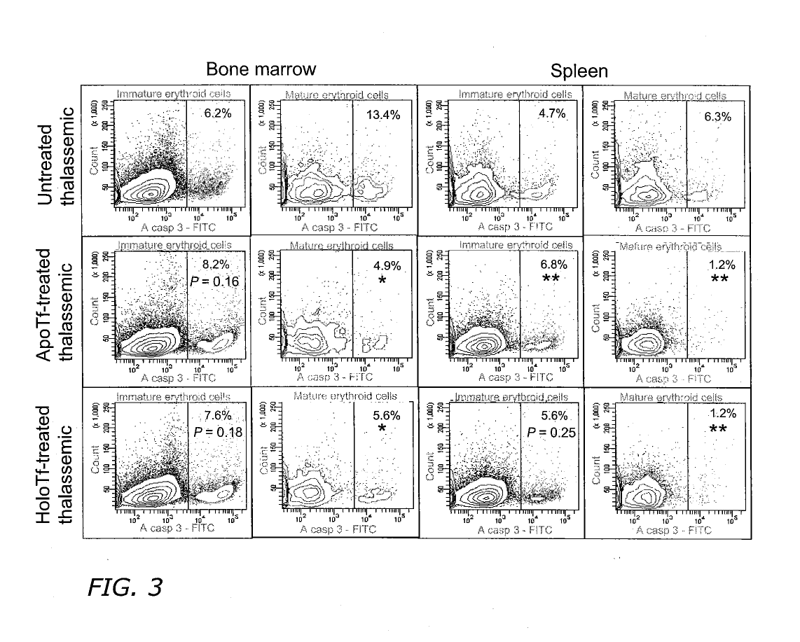

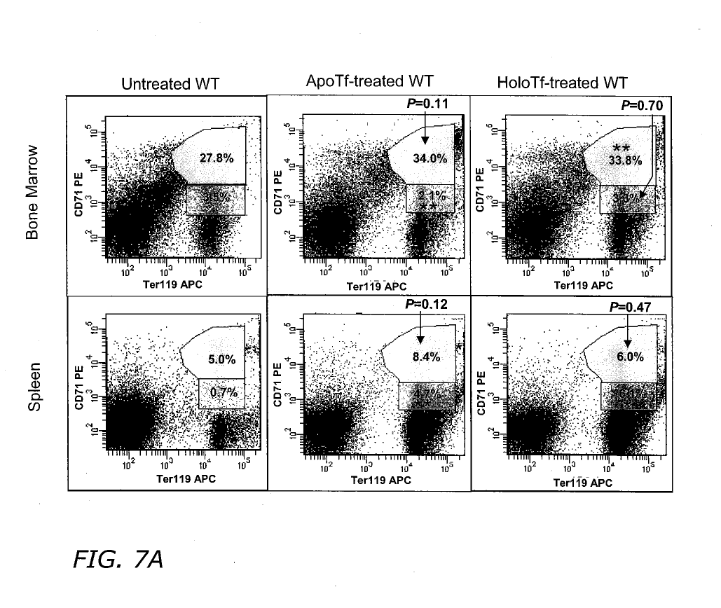

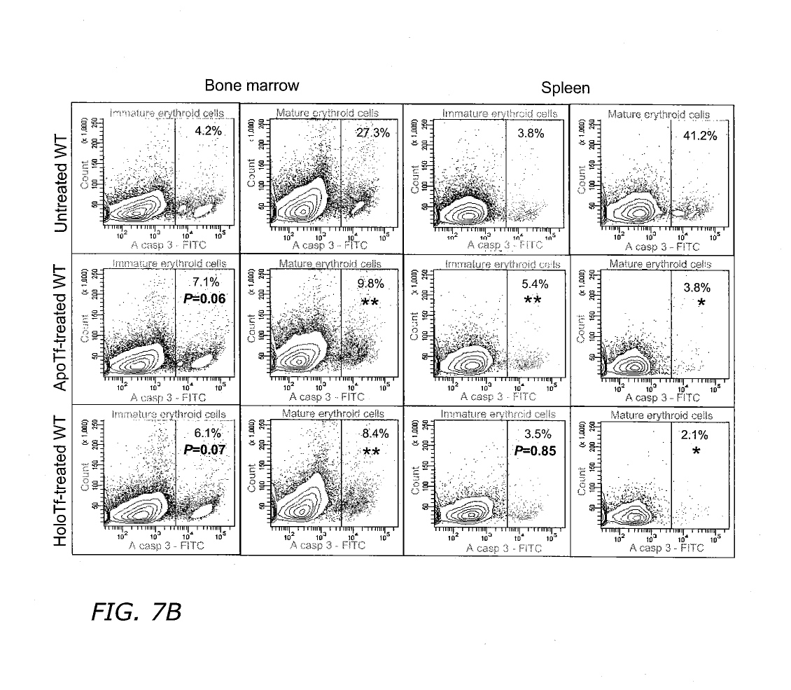

The distribution of erythroid precursors in the spleen and bone marrow shifted to a higher proportion of mature (TER119+CD71−) relative to immature (TER119+CD71+) cells (FIG. 2C). The TER119+CD71− cell population represents proerythroblasts and basophilic erythroblasts, and the TER119+CD71+ cell population represents polychromatophilic and orthochromatophilic erythroblasts, as determined by cytospin analysis of the sorted cells (data not shown). In transferrin-treated WT mice, increased mature erythroid precursors were observed only in the spleen (FIG. 7A). Increased apoptosis of erythroid lineage cells was observed in a different mouse model of β-thalassemia intermedia, and thalassemic mice have a higher degree of apoptosis in erythroid precursors than WT mice. Therefore, it was tested whether erythroid precursor apoptosis was altered in transferrin-treated thalassemic mice. Transferrin treatment of thalassemic mice led to increased apoptosis in immature erythroid precursors and decreased apoptosis in mature erythroid precursors, as measured by activated caspase-3 (FIG. 3) and annexin V (data not shown). These findings, considered together with decreased reticulocytosis (Table 3), increased number of mature erythroid precursors and decreased number of immature erythroid precursors (FIG. 2C), suggest that, after transferrin treatment, fewer immature erythroid precursors are needed to maintain steady-state erythropoiesis because a higher proportion of those precursors develop to maturity, resulting in more effective erythropoiesis. A similar finding of decreased apoptosis of erythroid precursors was observed in transferrin-treated WT mice (FIG. 7C).

### Example 6

### Liver Hepcidin Expression is Enhanced and FPN-1 Levels Reduced

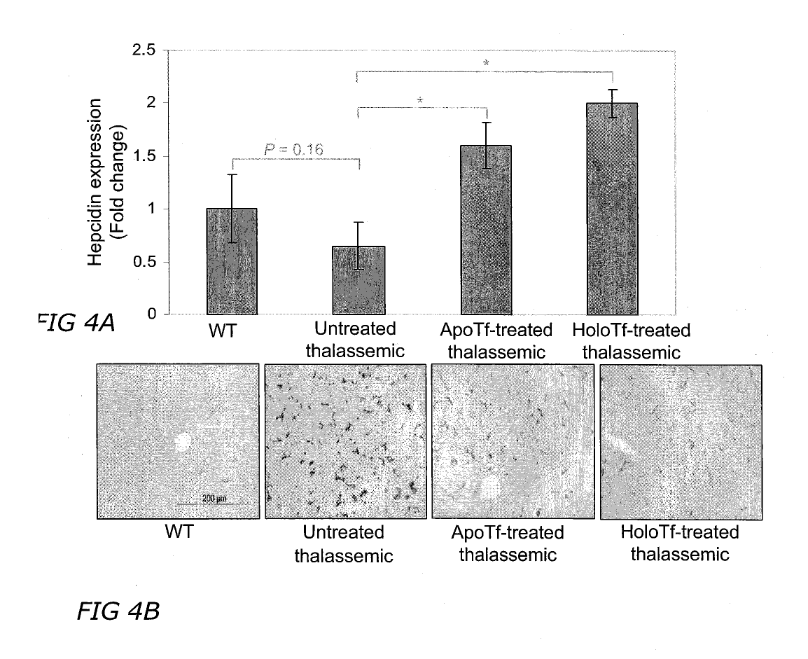

Hepcidin expression was higher in the livers of transferrin-treated than untreated thalassemic mice (FIGS. 4A and B). This increase in hepcidin is most likely due to diminished release from erythroid precursors of a suppressor of hepcidin function. No difference was observed in hepcidin expression in transferrin-treated relative to untreated WT mice (FIG. 6E). FPN-1, as measured by immunohistochemistry, was found on fewer Kupffer cells in the livers of transferrin-treated mice, whereas iron was observed by Perls' Prussian blue staining in a similar number of cells. Both increased hepcidin expression and decreased FPN-1 levels would be expected to result in reduced iron recycling. Reduced FPN-1 expression would be expected to result in less iron absorption by duodenal enterocytes.

### Methods for Examples 1-6

### Mice

WT (C57BL/6J; C57) and thalassemic mice (mixed background) were purchased from Jackson Laboratories. Thalassemic mice were backcrossed onto a C57 background. Age and gender-matched 9-10 month old thalassemic and WT mice were used. All mice were bred and housed in the Lindsley F. Kimball Research Institute Animal Facility under AAALAC guidelines. The experimental protocols were approved by the facilities Animal Institute Committee. All mice had access to food and water ad libitum.

Transferrin Regimen.

Mice were injected daily for a total of 60 days; this course was intended to represent a chronic state of increased transferrin in the circulation. The optimum dose of 10 mg transferrin per day (400 mg/kg/day) was determined by dose escalation experiments. Daily injections were employed in light of the 34-40 hr half-life of endogenous transferrin in mice and on the basis of prior experiments in hypotransferrinemic mice. Both apoTf and holoTf transferrin preparations were used. Additional mice were treated with a 10 day course.

Transferrin Production/Purification.

ApoTf and holoTf were prepared from human plasma by a process suitable for large scale manufacturing of transferrin for investigational human clinical use as previously described (von Bonsdorff, L. et al. Biologicals. 29:27-37, 2001). Briefly, transferrin was purified by Cohn fractionation and chromatographic techniques, and included steps to inactivate and remove potential adventitious viral agents. HoloTf was prepared via transferrin saturation or apoTf by removing excess iron. The iron content and the iron binding capacity were determined as described by von Bonsdorff. HoloTf was more than 90% iron saturated with less than a 7% iron binding capacity, whereas apoTf was less than 1% iron saturated with greater than 96% iron binding capacity. Both final products had a purity of over 98%, containing low levels of hemopexin and immunoglobulins as described by von Bonsdorff.

Hematopoietic Parameters.

RBC indices and reticulocyte counts were derived using a flow cytometry-based hematology analyzer, the Advia 120 Hematology System (Bayer Diagnostics) using specific equations intended to measure mouse specimens. Mouse RBCs collected via tail vein (40 μl) were suspended in saline containing EDTA.

LPI Determination.

The method was based on the oxidation of non-fluorescent dihydrorhodamine 123 (DHR) to fluorescent rhodamine 123 by reactive oxygen species, as described previously (Esposito, B. P. et al. Blood. 102:2670-77, 2003; Pootrakul, P. et al. Blood. 104:1504-10, 2004). Briefly, DHR (50 μM) and ascorbate (40 μM) were added to each serum sample and samples were tested under 2 conditions: with or without 50 μM deferiprone. The slopes of rate of increase of rhodamine 123 fluorescence were obtained in a fluorescent plate reader and the LPI concentration (μM) calculated using known iron concentration standards (0-5 μM Fe:nitrilotriacetate at 1:10 ratio).

RBC Lifespan.

Sulfo-N-hydroxysuccinimide biotin (EZ-Link, Pierce) was injected into the tail vein on day 0 (t=0). RBC samples (2-5 μl of tail vein blood) were analyzed after incubation with fluorescein isothiocyanate-conjugated avidin (Vector Laboratories) as described previously (de Jong, K. et al. Blood. 98:1577-84, 2001; Beauchemin, H. et al. J Biol Chem. 279:19471-80, 2004). The number of biotinylated RBCs was determined using flow cytometry (FACScan, Becton Dickinson) at t=0 and at various intervals during a 60 day period. Survival data were fitted to A(t)=A_{0[}1−(t/T)]e^{-kt}, in which t=time, T=time at which A(t) is 0, A₀=A(t) at t=0, and k=constant.

Analysis of RBC Membrane-Associated Globin Precipitate.

Equal numbers of RBCs were lysed and membranes washed. Membrane skeleton fractions were prepared as described previously (Sorensen, S. et al. Blood. 75:1333-36, 1990; Kong, Y. et al. J Clin Invest. 114:1457-66, 2004). Briefly, membrane lipids were removed, globins were dissolved and fractionated by Triton-acetic acid-urea (TAU) gel electrophoresis. After staining, the images were acquired on Gel logic 200 Imaging System using Kodak Molecular Imaging software (version 4.0.4).

Quantitative Real-Time Polymerase Chain Reaction (Q-PCR).

RNA from liver was prepared using the RNeasy Kit (Qiagen Sciences) according to the manufacturer's instructions. Single-pass cDNA was synthesized using 5 μg total RNA, Superscript III RNase H⁻ reverse transcriptase (Invitrogen), and anchored oligo dT. Q-PCR analysis was performed using the ABI 7900HT Sequence Detection System in a 384-well set-up (Applied Biosystems) with SYBR green. Hepcidin mRNA was amplified using primers for mouse hepcidin 1. Control GAPDH mRNA was amplified using primers GAPDH F and GAPDH R (Qiagen). mRNA concentrations of the target gene (Hamp1) were normalized to a reference stable housekeeping gene (GAPDH).

Fluorescence-Activated Cell Sorting Analysis and Quantification.

Bone marrow and spleen cells were incubated with anti-mouse TER119-allophycocyanin (APC) and CD71-phycoerythrin (PE). Apoptosis was detected using Annexin V-fluorescein or activated caspase 3-fluorescein (both from Molecular Probes). Necrotic cells were identified with 7-Amino-actinomycin D (7AAD, BD Pharmingen). Erythroid precursors were selected by gating and analyzed using CD71 and TER119. Results were acquired on a flow cytometer FACSCalibur (Becton Dickinson) using CellQuest Pro version 3.3 software.

Statistical Analyses.

All data are reported as mean±standard error. Analysis for statistically significant differences was performed using the Student unpaired t-test. A parametric decreasing exponential nonlinear mixed effects model was used to estimate the median survival of RBC using maximum likelihood.

### Example 7

### Exogenous Transferrin Injections in Splenectomized Mice

The previous experiments demonstrated that apotransferrin (ApoTf) injections ameliorate anemia, extramedullary erythropoiesis, splenomegaly and iron overload in a mouse model of β-thalassemia intermedia. Although the number of red blood cells in circulation and total hemoglobin increased, the MCH, or the amount of hemoglobin in each red blood cell decreased. These results suggest that anemia in β-thalassemia is a consequence of excess intracellular heme in developing erythroblasts. Splenectomy is a significant clinical intervention in patients with β-thalassemia syndromes, often providing at least a temporary reprieve from escalating transfusion. The data presented here reveals the effect of ApoTf on splenectomized mice and enables an evaluation of its effect on erythroid maturation and cell surface transferrin receptor 1 (TfR1) expression. Exogenous transferrin injections reduce MCH by decreasing surface TfR1 expression to improve erythroid maturation.

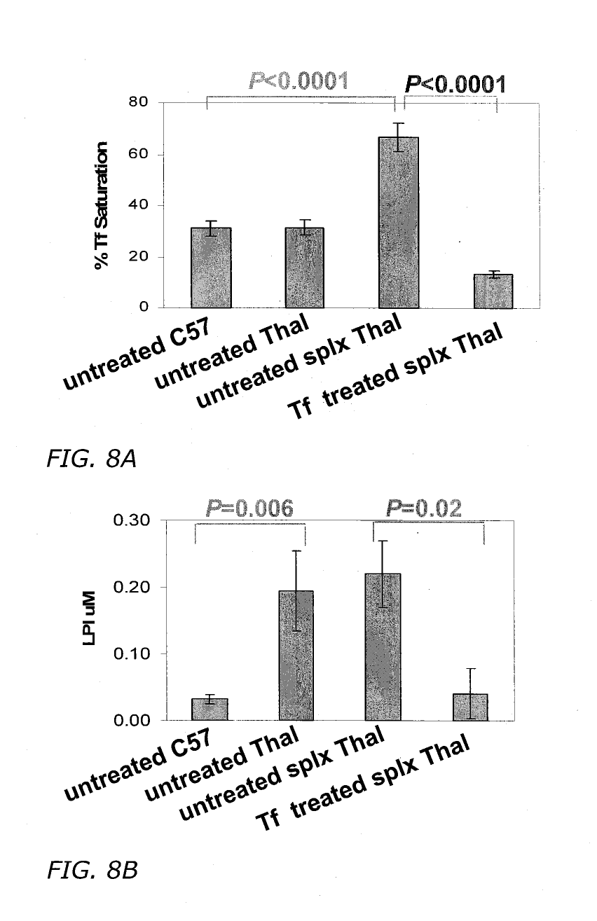

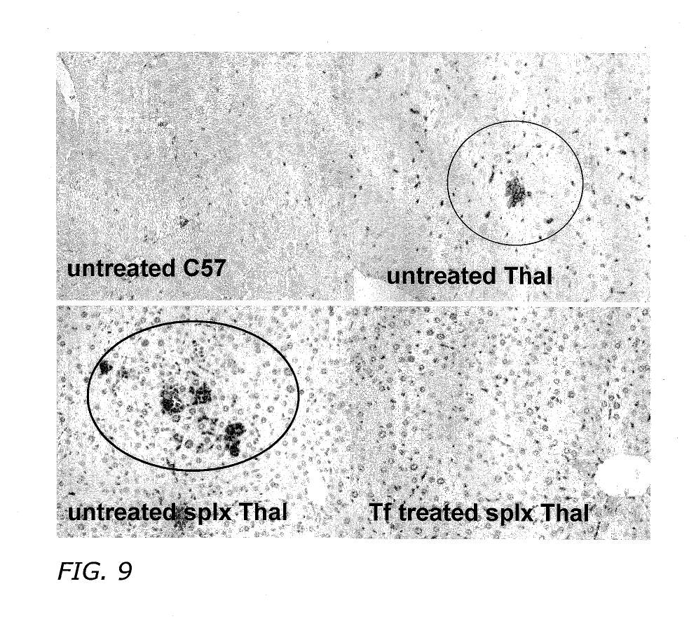

Seven-month old female splenectomized (splx) thalassemic (Thal) mice were compared both before and after ApoTf administration to age-matched female non-splx Thal mice and C57BL/6 controls. The mice received IP injections of 10 mg (200 μL) of ApoTf daily for 20 days. As in non-splx Thal mice, human ApoTf maintained function in mouse circulation, reduced transferrin saturation (FIG. 8A) and normalized LPI levels (FIG. 8B) in splx Tal mice. Extramedullary erythropoiesis in the liver increased in splx Thal mice and disappeared after ApoTf treatment (FIG. 9)

Furthermore, ApoTf injections improved red blood cell parameters, resulted in smaller RBCs with lower MCH and reduced reticulocytosis in splx Thal mice (Table 5).

TABLE 5

| | RBC | Hb | MCV | MCH | MCHC | Retics |
|---|---|---|---|---|---|---|
| | (×10⁶ cells/L) | (g/dL) | (fL) | (pg) | (g/dL) | (×10⁹ cells/L) |
| C57 | 10.5 | 15.1 | 42.1 | 14.3 | 34.0 | 225 |
| Untreated Thal | 8.4* | 8.5* | 35.9* | 10.1* | 28.3* | 2664* |
| Untreated splx Thal | 6.9* | 7.5* | 37.3 | 11.1* | 29.8* | 1470* |
| Tf treated splx Thal | 13.4* | 11.3* | 28.7* | 8.6* | 30.3 | 538* |

*P < 0.001 untreated Thal vs. C57;
*P < 0.001 untreated splx Thal vs. untreated Thal;
*P < 0.001 Tf-treated vs. untreated splx Thal

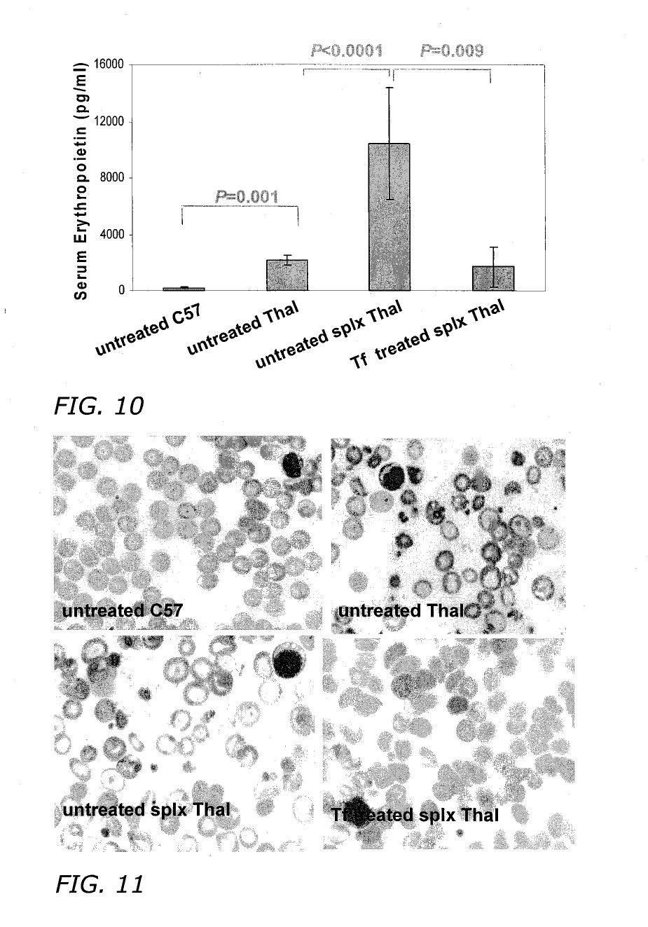

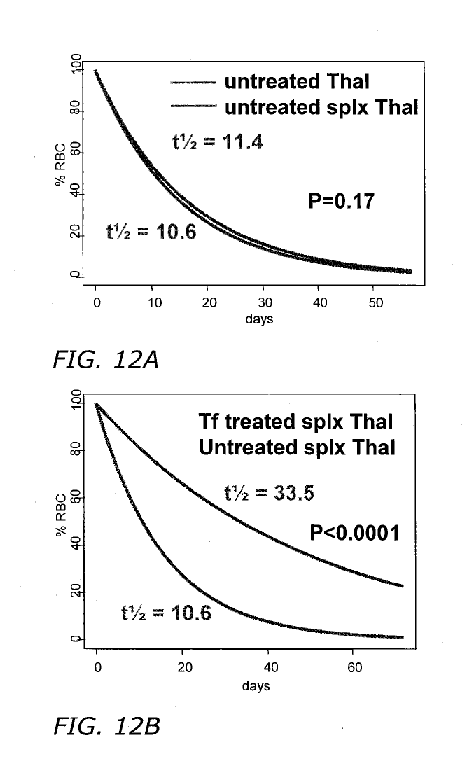

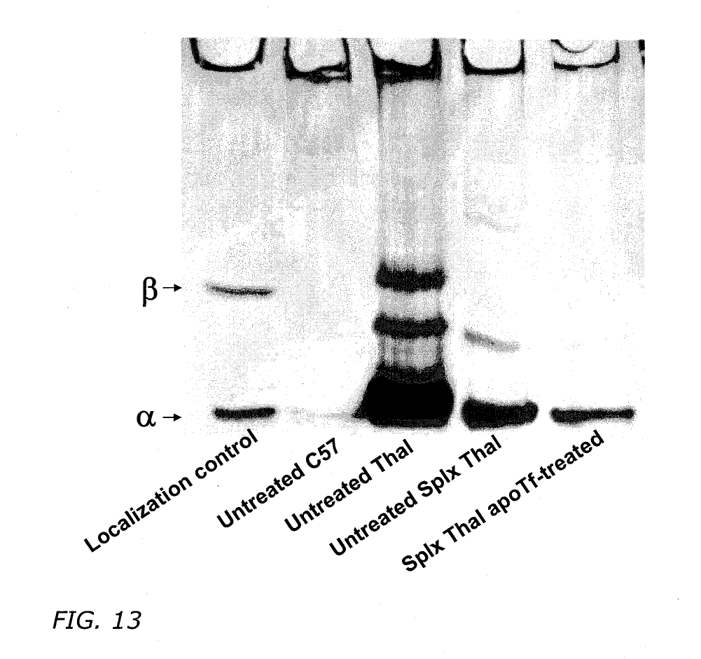

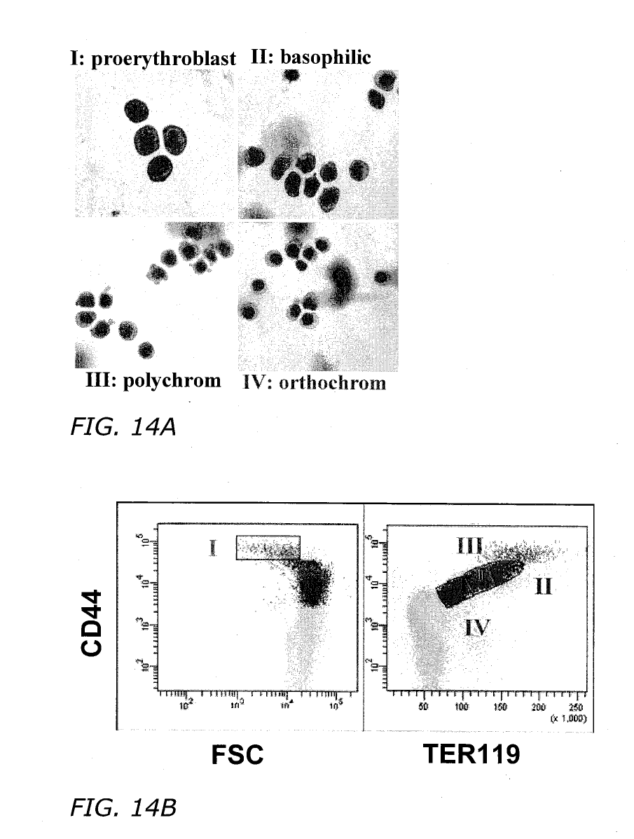

Serum erythropoietin increased in splx Thal mice and returned to pre-splenectomized levels after ApoTf treatment (FIG. 10). Additionally, ApoTf treatment improved RBC morphology in splx Thal mice (FIG. 11), normalized RBC lifespan (FIGS. 12A and B), and reduced α-globin precipitation on RBC membranes (FIG. 13) in splx Thal mice. ApoTf treatment normalized disordered erythropoiesis in early stages of terminal erythroid differentiation in Thal mice pre- and post-splenectomy (Table 6). This data was confirmed by cytospin (FIG. 14A) and flow cytometry analysis of forward scatter or TER119 expression versus CD44 expression (FIG. 14B).

TABLE 6

| | | | Untreated | ApoTf-treated |
|---|---|---|---|---|
| | C57 | Thal | Splx Thal | splx Thal |
| I. proerythroblast (%) | 1.7 | 2.7* | 3.4 | 2.1 |
| II. basophilic (%) | 3.4 | 10.0* | 10.6 | 4.3** |
| III. polychromatophilic (%) | 6.9 | 15.5* | 18.8 | 8.9** |
| IV. orthochromatophilic (%) | 13.9 | 38.8* | 34.5 | 19.4** |

*P < 0.01 vs. C57;
**P < 0.02 vs. untreated splx Thal

The MFI of CD71 (TfR1), in all stages of erythroid differentiation, increased with splenectomy and decreased in apotransferrin-treated splx Thal mice (Table 7).

TABLE 7

| Erythroid differentiation stage | | | Untreated | ApoTf-treated |
|---|---|---|---|---|
| (×1000) | C57 | Thal | Splx Thal | splx Thal |
| proerythroblast | 31 | 73* | 105** | 60*** |
| basophilic | 29 | 43 | 91** | 52*** |
| polychromatophilic | 22 | 39* | 63** | 39*** |
| orthochromatophilic | 17 | 28* | 43** | 28*** |

*P < 0.001 vs. C57;
**P < 0.02 vs. pre-splx Thal;
***P < 0.01 vs. untreated splx Thal

These findings expand the use of transferrin injections to ameliorate disease in thalassemia to splenectomized individuals. These data confirm that apotransferrin injections result in more iron deficient erythropoiesis by decreasing surface TfR1 which causes a reduction in heme synthesis and supports the decrease in MCV and MCH in RBCs in apotransferrin-treated Thal mice.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method for reducing spleen size in a patient with thalassemia comprising administering at least one course of transferrin to said patient and thereby reducing the size of said spleen in said patient.

2. The method of claim 1 wherein said thalassemia is β-thalassemia intermedia.

3. The method of claim 1 wherein said thalassemia is β-thalassemia major.

4. The method of claim 1 wherein said course comprises a plurality of doses of transferrin.

5. The method of claim 4 wherein said course comprises administering doses of transferrin for 7-21 days.

6. The method of claim 4 wherein said dose of transferrin is administered every day during said course.

7. The method of claim 4 wherein said dose of transferrin is administered every other day during said course.

8. The method of claim 4 wherein said course comprises administering doses of transferrin every day for a certain number of days and every other day for a certain number of days.

9. The method of claim 1 wherein said course is repeated at an interval selected from the group consisting of every other month, every third month, and every fourth month.

10. The method of claim 4 wherein each dose comprises about 25-150 mg/kg of transferrin.

11. The method of claim 1 wherein said transferrin is apotransferrin.

12. The method of claim 1 wherein said transferrin is human transferrin.
